(19) United States
(12) Plant Patent Application Publication
(10) Pub. No.: US 2016/0324047 P1
(43) Pub. Date: Nov. 3, 2016

(54) STRAWBERRY PLANT NAMED 'STEPHANIE'

(50) ;

(71) Applicant: John LARSE, Aptos, CA (US)

(72) Inventor: John LARSE, Aptos, CA (US)

(73) Assignee: Sweet Darling Sales, Inc., Aptos, CA (US)

(21) Appl. No.: 14/545,382

(22) Filed: Apr. 29, 2015

(51) Int. Cl. A01H 5/00

(52) U.S. Cl. PLT/208

(57) ABSTRACT

The present invention provides a new and distinct strawberry varieties designated as ‘Stephanie’ (a.k.a. ‘Stefanie’ or ‘104435’).

### LATIN NAME OF THE GENUS AND SPECIES

Fragaria×ananassa

### VARIETAL DENOMINATIONS

‘Stephanie’ (a.k.a. ‘Stefanie’ or ‘104435’)

### BACKGROUND OF THE INVENTION

The present invention relates to a new and distinct strawberry variety designated as ‘Stephanie’ (a.k.a. ‘Stefanie’ or ‘104435’).

‘Stephanie’ (a.k.a. ‘Stefanie’ or ‘104435’) is the result of a controlled-cross between a female parent cultivar designated 1366 and a male parent cultivar designated 1285 (both unpatented, proprietary cultivars) made by the Inventor and was first fruited in Watsonville, Calif. growing fields. Following selection and during testing, the plant was originally designated ‘104435’ and subsequently named ‘Stephanie’.

The new variety was asexually reproduced via runners (stolons) by the inventor at Watsonville, Calif. Asexual propagules from the original source have been tested in Watsonville growing fields and to a limited extent, grower fields in high elevation. The properties of the variety were found to be transmissible by such asexual reproduction. The cultivar is stable and reproduces true to type in successive generations of asexual reproduction.

### BRIEF SUMMARY OF THE INVENTION

This invention relates to a new and distinctive strawberry cultivar designated as ‘Stephanie’ The cultivar was primarily adapted to the climate and growing conditions of the central coast of California. This region provides the necessary temperatures required for it to produce a strong vigorous plant and to remain in fruit production from March through October. The nearby Pacific Ocean provides the needed humidity and moderate day temperatures and evening chilling to maintain fruit quality for the production months.

The following traits and photographs in combination distinguish strawberry variety ‘Stephanie’ from known strawberry varieties. In addition, the new cultivar was confirmed to be a unique strawberry germplasm when tested against the California Seed & Plant Lab, Inc. (Elverta, Calif.) database using Short Sequence Repeats (SSRs). Plants for the botanical measurements in the present application were grown as annuals. Any color references are made to The Royal Horticultural Society Colour Chart, 1995 Edition, except where general terms of ordinary dictionary significance are used.

The fruit produced by the new cultivar is attractive and of excellent quality.

### DESCRIPTION OF THE DRAWINGS

The accompanying color photographs depict various characteristics of the cultivar as nearly true as possible to make color reproductions.

FIG. 1 shows ‘Stephanie’ plants with ripe and near-ripe fruits.

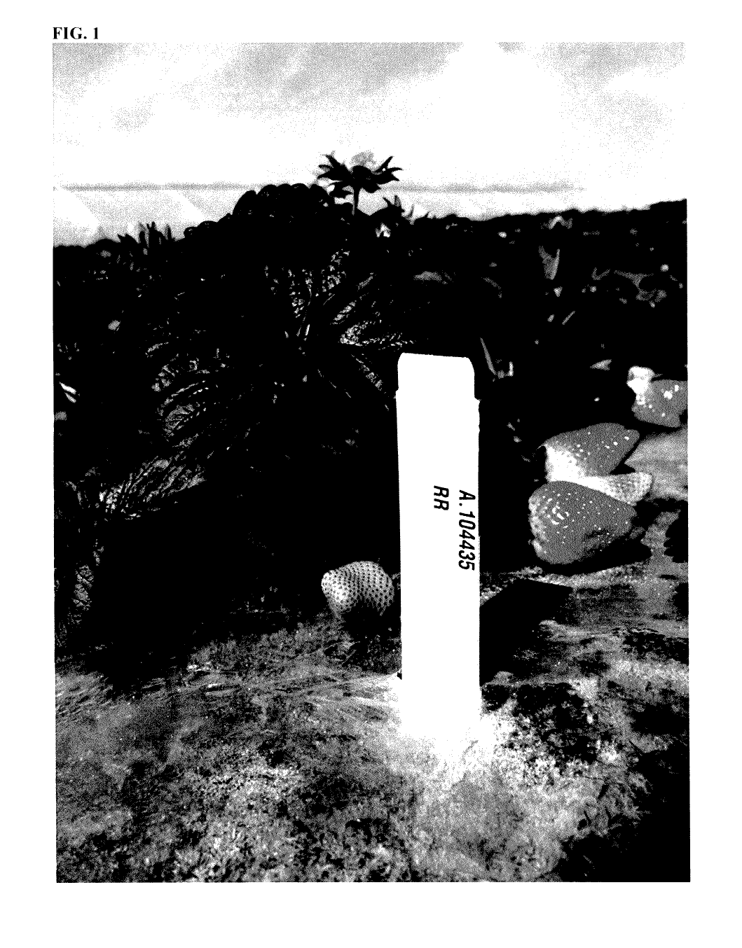

### DETAILED DESCRIPTION OF THE INVENTION

### ‘Stephanie’ (a.k.a. ‘104435’)

This invention relates to a new and distinctive day-neutral type cultivar designated as ‘Stephanie’. It is primarily adapted to the climate and growing conditions of the central coast of California. This region provides the necessary temperatures required for it to produce a strong vigorous plant and to remain in fruit production from April through September. The nearby Pacific Ocean provides the needed humidity and moderate day temperatures and evening chilling to maintain fruit quality for the production months.

The following traits in combination distinguish strawberry variety ‘Stephanie’ from the known strawberry varieties. Plants for the botanical measurements in the present application were grown as annuals. In the following description, color references are made to The Royal Horticultural Society Colour Chart, 1995 Edition, except where general terms of ordinary dictionary significance are used.

‘Stephanie’ has not been observed under all possible environmental conditions, and the phenotype may vary significantly with variations in environment.

Hybrid Patent Data Sheet
‘Stephanie’ (a.k.a. ‘104435’)

| Plant | |
|---|---|
| Height (mm) | 320 |
| Diameter (mm) | 540 (the entire plant structure, including |
| | the “superstructure” of petioles, leaves, |
| | flowers) |
| | 50 (inner “base” or “heart” structure of |
| | the plant, i.e. the mature apical and axial |
| | crowns of the strawberry plant where |
| | petioles, flowers and runners are |
| | attached) |
| Terminal Leaf | |
| Length (mm) | 98 |
| Width (mm) | 110 |
| Blistering | |
| Petiole | |
| Length (mm) | 94 |
| Width (mm) | 3 |
| Petiolule | |
| Length (mm) | 210 |
| Width (mm) | 5 |
| Stolon | |
| Count (mm) | 522 |
| Diameter (mm) | 4 |
| Inflorescence | |
| Petal/flower | |
| Corolla (mm) | 16 |
| Calyx (mm) | 10 |
| Petal length (mm) | 17 |
| Petal width (mm) | 6 |
| Sepal length (mm) | 20 |
| Sepal width (mm) | 8 |
| Peduncle | |
| Stipules | |
| Fruit | |
| Fruit/truss | |
| Length (mm) | 50 |
| Width (mm) | 40 |
| Core length (mm) | 40 |
| Core width (mm) | 17 |

Achenes/fruit (measured only once)

| | |
|---|---|
| Glossiness | N/A |

1. A new and distinct cultivar of strawberry plant named ‘Stephanie’ substantially as shown and described herein.
